(19) United States
(12) Patent Application Publication
(10) Pub. No.: US 2011/0282417 A1
(43) Pub. Date: Nov. 17, 2011

(54) COCHLEA INSERTABLE HEARING PROSTHESIS AND CORRESPONDING SYSTEM AND METHOD

(76) Inventors: Tobias Moser, Goettingen (DE); Gerhard Hoch, Gleichen (DE)

(21) Appl. No.: 12/779,291

(22) Filed: May 13, 2010

(51) Int. Cl. A61F 11/04

(52) U.S. Cl. 607/92

(57) ABSTRACT

A hearing prosthesis insertable fully or partially into the human cochlea provides for hearing support capabilities to persons with degraded hearing capability or without hearing capability. The hearing prosthesis emits controlled light signals for stimulation of hearing neurons. Using light sources, the transfer of audio signals to a person is realized optically, as opposed to transfer through the use of electrodes. The light signals are controlled in terms of duration, intensity and repetition frequency from the light sources.

The invention is related to a hearing prosthesis which is insertable in the human cochlea according to claim 1. The invention is further related to a hearing prosthesis system according to claim 18 and a method for operating a hearing prosthesis according to claim 19. More generally the invention is related to support devices for persons with a degraded capability of hearing or without hearing capability.

In this area the so called cochlea implants (CI) have become a widely used solution. A typical cochlea implant comprises a number of incitation electrodes which are implanted into the cochlea of a human by surgery. The number of electrodes which can be implanted is limited due to the available space in the cochlea. Typically, a maximum of about ten independent excitation channels can be realized. As a result of this limited number of excitation channels, the hearing quality is not optimal. In several situations of daily life the hearing capability provided by a typical cochlea implant is overstrained by complex ambient audio signals and disturbing noise.

It is therefore an object of the invention to propose a device and a method which provides persons with reduced or no hearing capabilities with a hearing support having an improved hearing quality.

This object is achieved by the hearing prosthesis according to claim 1, the hearing prosthesis system according to claim 18 and the method for operating a hearing prosthesis according to claim 19. The dependent claims comprise further advantages improvements of the invention.

An in the human cochlea insertable hearing prosthesis for use in the human cochlea with signal emitting means for the stimulation of hearing neurons is proposed which comprises the following features:
a) the signal emitting means comprise light sources,
b) the hearing prosthesis comprises a microelectronic control device, which is arranged for the control of at least one of the intensity, the duration and the repetition frequency of light emission of the light sources.

By the introduction of light sources the transfer of audio signals can be realized by optical devices instead of the currently used electrodes. The use of optical signal transmission allows for the increase of available excitation channels by a factor e.g. 100, which results in a significant increase in hearing quality. Further, the proposed hearing prosthesis is capable of being inserted into the human cochlea. This includes complete or partial insertion into the cochlea. However, at least the light sources shall be completely insertable into the cochlea. This allows for a direct emission of light from the light sources on the hearing neurons. The insertable hearing prosthesis can therefore be manufactured with a compact, small design because no further light transferring means, like e.g. fiber-optic light guides, are required.

The light sources provide for a direct emission of light on the intented location in the human cochlea. No light guiding elements are required, like optical fibres or the like, which results in a very compact, small design of the hearing prosthesis. The light sources, e.g. micro-LEDs, advantageously can be placed directly in the human cochlea.

Further, the hearing prosthesis comprises an integrated microelectronic control device. The control device is arranged for controlling the light sources. In particular, at least one of the intensity, the duration and the repetition frequency of light emission of the light sources can be controlled by the microelectronic control device. The microelectronic control can control one, more or each of the intensity, the duration and the repetition frequency of light emission of the light sources. At least that part of the microelectronic control device which is directly connected with the light sources shall be located in proximity of the light sources. It is advantageous that also the microelectronic control device is completely or partly insertable into the cochlea.

It is advantageous to place the hearing prosthesis directly in the human cochlea. Through the light sources light can be directly emitted on the spiral ganglion neurons in the cochlea.

It is advantageous to control the intensity and/or the duration of light emission of the light sources according to audio information to be reproduced.

In a further advantageous embodiment at least a part of the light sources is arranged for the emission of light in the visible wave length range, in particular in the wave length range from 380 to 770 nm. The use of light, in particular in the visible wave length range from 380 to 770 nm, allows for better signal transfer by improved spiral ganglion neuron activation, inhibition and deactivation.

In a further advantageous embodiment the hearing prosthesis comprises measuring electrodes which are arranged for capturing stimulation signals of the spiral ganglion neurons. The microelectronic control device is connected with the measuring electrodes. Further, the microelectronic control device is arranged for control of at least one of the intensity, the duration and the repetition frequency of the light emission of the light sources in dependence of the signals received from the measuring electrodes. This allows for a closed loop control process of the light sources, which leads to an improved hearing quality. In particular, by using the feedback provided from the measuring electrodes the light emission can be adaptively controlled by the microelectronic control device.

In a further advantageous embodiment the light sources are formed as a chain of a plurality of illumination segments, which are flexibly connected with each other. This has the advantage that a relatively high number of individual light source bodies of the light sources can be directly inserted into the human cochlea. Through the flexibility of the chain of illumination segments the device can be adapted to the form of the cochlea. In a further advantageous embodiment the flexible connection of the illumination segments is used as signal transmission path for the light sources control signals of the microelectronic control device.

In a further advantageous embodiment, the illumination signals are arranged with displacement between neighbouring segments. The neighbouring segments are overlapping. For example, the illumination segments can be arranged like a “double chain”. This allows for a continuous light emission density throughout the extent of the light sources.

In a further advantageous embodiment the light sources comprise a plurality of light source bodies which are independently from each other controllable by the microelectronic control device. For example, in an advantageous implementation of the invention a number of 1000 to 2000 light source bodies can be provided. Through the independent control by the microelectronic control device the light source bodies can be individually activated and deactivated, thereby providing a high number of independent excitation channels for incitation of hearing neurons. This provides for further improvements in hearing quality.

In a further advantageous embodiment one, a plurality or all light source bodies are each equipped with own light focusing means. The light focusing means can be implemented, e.g., by a parabolic reflector or by a lense. By use of the light focusing means the precision of light emission to designated points in the cochlea can be increased, thereby further improving the hearing quality.

In a further advantageous embodiment one, a plurality or all light source bodies are each controllable through a controllable current source. The controllable current sources provide electrical energy to the light source bodies. This allows for a further improved hearing quality through linearization of any nonlinear characteristic diagram of a light emission body, e.g. LED, through its current source.

In a further advantageous embodiment the light sources comprise micro-LEDs. An LED is a light emitting diode. By use of micro-LEDs, a high number of independent light source bodies can be implemented in small size, so that a hearing prosthesis which is insertable in the human cochlea can be equipped with the mentioned high number of independent light source bodies which represent the independent excitation channels.

In a further advantageous embodiment the light sources are arranged for light emission with different wave lengths. For example, it is possible to equip the light sources with a combination of blue and yellow LEDs. Depending on the requirements and light reception characteristics in the cochlea, further wave lengths which correspond to further colours can be implemented. It is advantageous to use only light sources which emit light in the visible wave length range.

In a further advantageous embodiment the light sources are arranged for the emission of blue, yellow and/or green light. In a further advantageous embodiment, the control device is arranged for controlling the light sources to emit blue light for the activation of a hearing neuron, to emit yellow light for the inhibition of a hearing neuron and to emit green light for the deactivation of a hearing neuron. This provides for a fast signal transfer and signal acceptance by the hearing neurons, which allows for further improvement of the hearing quality.

In a further advantageous embodiment the light sources comprise gallium nitride LEDs. Gallium nitride LEDs provide for blue light and can be implemented with very small dimensions, e.g. 10 to 40 μm per LED.

In a further advantageous embodiment the light sources and the control device are produced through CMOS gallium nitride flip-chip bonding. This allows for the production of hearing prosthesis with very small overall dimensions and with flexibility between illumination segments, so that a highly efficient hearing prosthesis with, a size can be produced which fits into the human cochlea.

In a further advantageous embodiment the light sources comprise line- and/or matrix-shaped arrangements of a plurality of light source bodies.

In a further advantageous embodiment the maximum length of the light sources is less than 30 mm.

A hearing prosthesis, system is proposed which comprises:
a) a hearing prosthesis of the aforementioned type, whereas the hearing prosthesis further comprises receiving means, for receiving hearing excitation signals,
b) a microphone device for receiving ambient acoustic signals,
c) a transformation unit connected to the microphone device, whereas the transformation unit is arranged for transforming the signals received from the microphone device into hearing excitation signals,
d) a transmission device connected to the transformation unit, whereas the transformation unit is arranged for transmitting the hearing excitation signals through the transmission device to the receiving device.

This system allows for supplying person with reduced or no hearing capabilities with the ability of hearing in normal daily life situations with an improved hearing quality compared to prior art devices.

In a further advantageous embodiment the transformation unit is arranged for the supply of electrical energy the human cochlea insertable hearing prosthesis. In a further advantageous embodiment the electrical energy is supplied by wireless transfer, e.g. through the transmission device which can comprise a radio communication unit.

An advantageous method for operating an in the human cochlea insertable hearing prosthesis of the aforementioned type provides for a control of at least one of the intensity, the duration and the repetition frequency of light emission of the light sources according to audio information to be reproduced.

In a further advantageous embodiment the hearing incitation signals are received from a transmission device and at least one of the intensity, the duration and the repetition frequency of light emission of the light sources is controlled according to the received hearing excitation signals.

In a further advantageous embodiment for the activation of a hearing neuron blue light is emitted, for the inhibition of a hearing neuron yellow light is emitted and for the deactivation of a hearing neuron green light is emitted.

In a further advantageous embodiment independent light source bodies of the light sources are individually controlled in dependence from the frequency range of the audio information to be reproduced. For example, the audio information to be reproduced can be provided in the frequency domain. From the signals in the frequency domain several parts can be assigned to certain light source bodies.

In a further advantageous embodiment stimulation signals of the spiral ganglion neurons are received by measuring electrodes and fed to the hearing prosthesis comprises which are arranged for capturing, whereas the hearing prosthesis controls at least one of the intensity, the duration and the repetition frequency of light emission of the light sources in dependence of the signals received from the measuring electrodes in a closed loop control process. This leads to an improved hearing quality.

On the side of the human which is the target for insertion of the hearing prosthesis the photosensitivity of the hearing neurons must be increased, so that the light emitted by the light sources can be utilized most efficiently. In an advantageous embodiment, light sensitive ion channels can be introduced into the spiral ganglion neuron using virus-dependent or virus-independent gene transfer. For example, channelrhodopsin-2 (ChR2) can be used as light sensitive ion channel. ChR2 is the light sensitive ion channel from green alga, which changes its conformation as a result of an activation of its retinal group by blue light. This leads to an opening of the channel and to non-selective cation conductivity. Further, halorhodopsin or bacteriorhodopsin can be introduced into the spiral ganglion neuron as a chloride pump. These pumps can be activated through yellow light. If activated, they inhibit neurons. Further ChR2 can be rapidly deactivated through green light. By means of this, depolarization of the hearing neurons can be terminated more rapidly than by just shutting of the blue light.

These and other advantageous of the invention are now described by means of the specific embodiments of the invention. In the drawings the following is depicted:

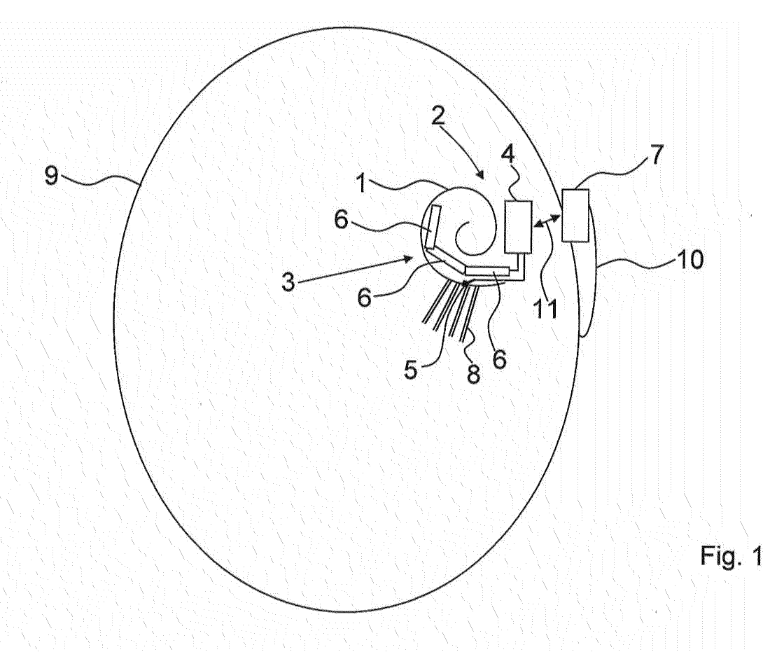

FIG. 1: a hearing prosthesis system and

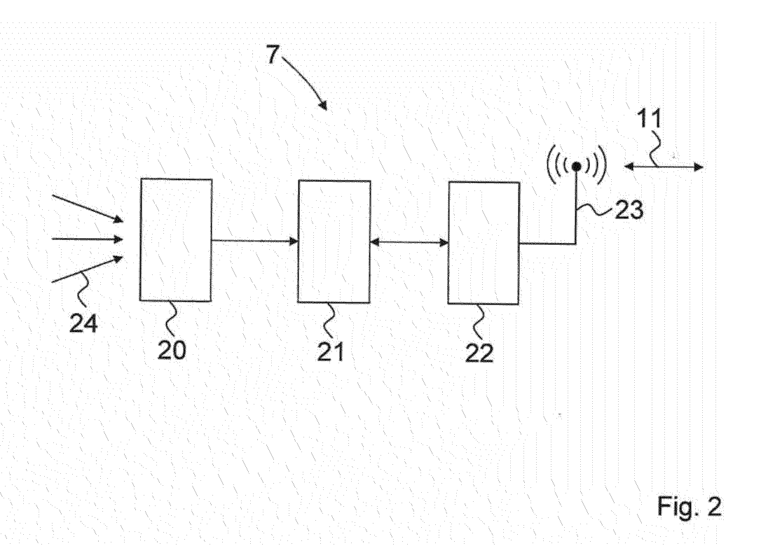

FIG. 2: a speech processing part of the hearing prosthesis system and

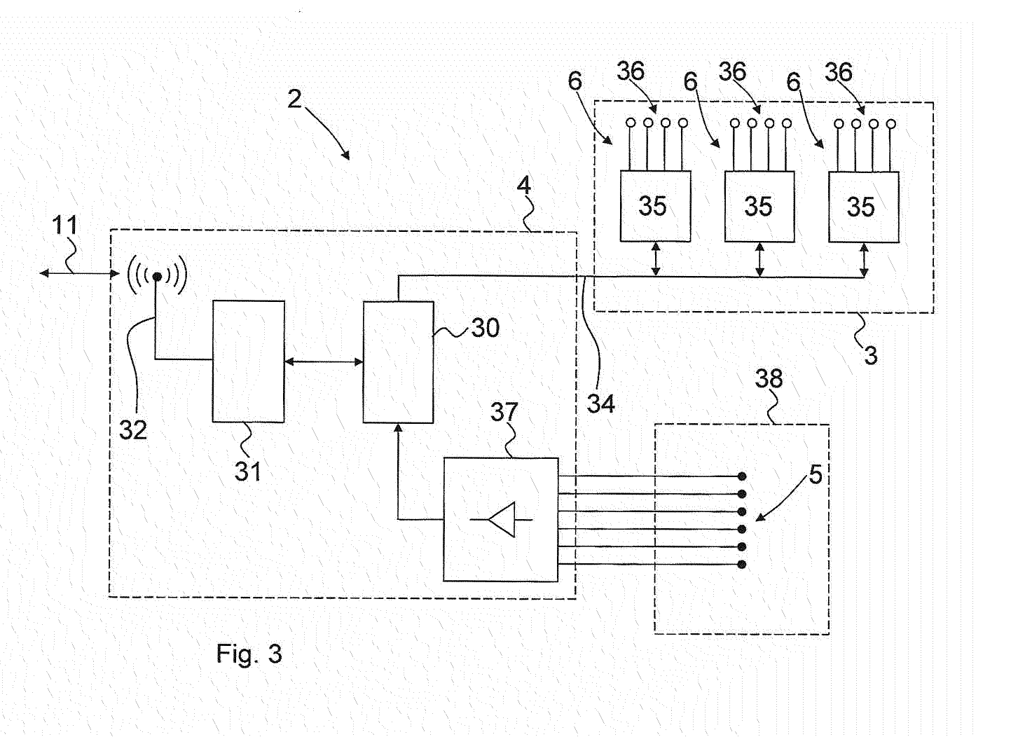

FIG. 3: an in the human cochlea insertable hearing prosthesis as a part of the hearing prosthesis system and

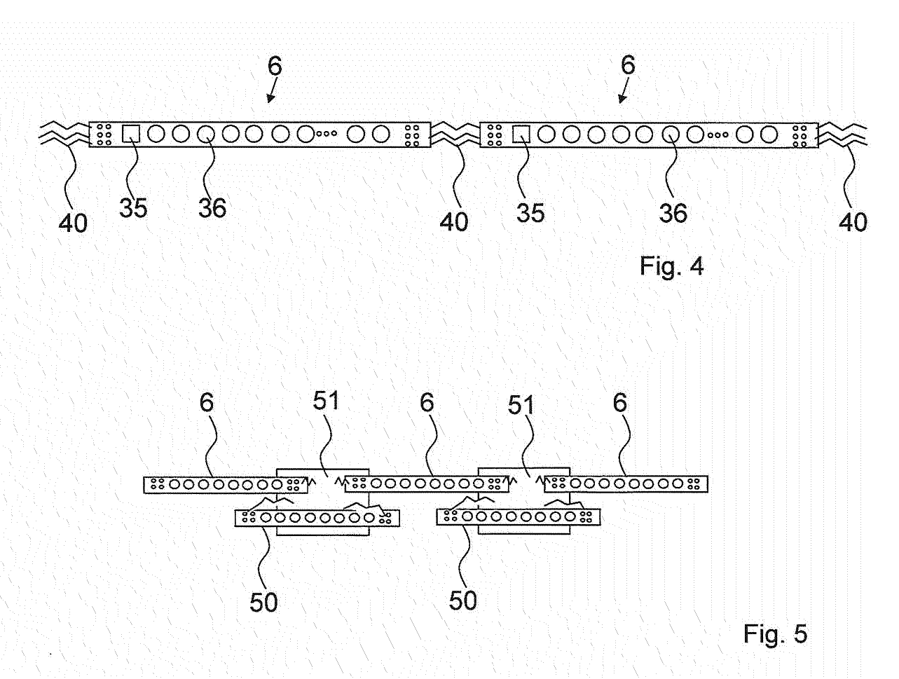

FIG. 4: a chain of illumination segments and

FIG. 5: a double chain of illumination segments and

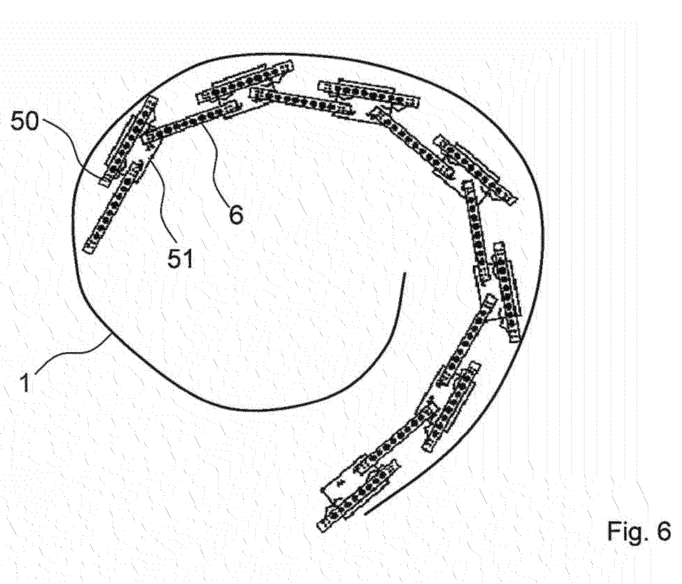

FIG. 6: a double chain of illumination segments inserted in a cochlea and

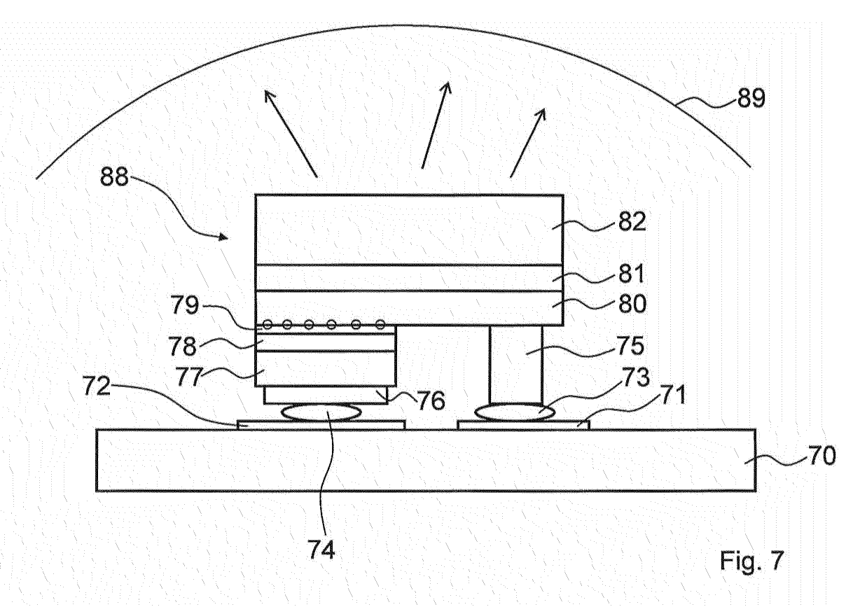

FIG. 7: a gallium nitride micro-LED and

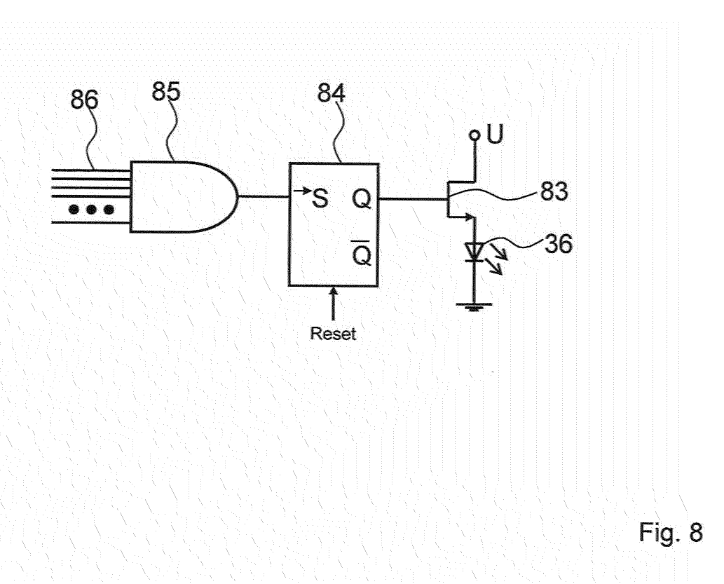

FIG. 8: a first embodiment of a control circuit for a LED and

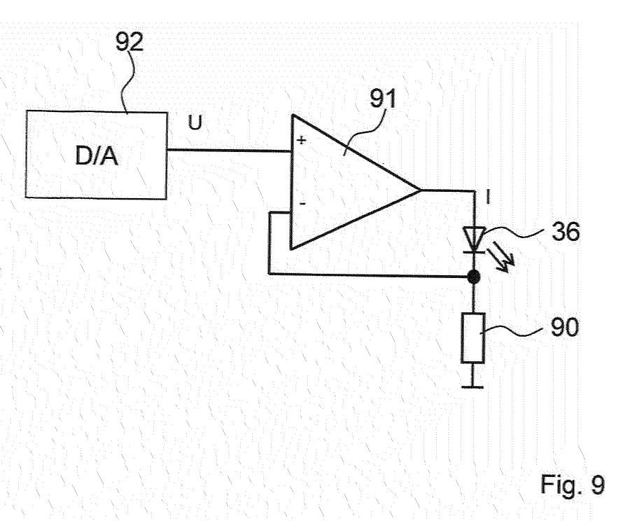

FIG. 9: a second embodiment of a control circuit for a LED.

The same reference numerals are used for the same elements throughout the drawings.

FIG. 1 shows a hearing prosthesis system applied to the head 9 of a human. The hearing prosthesis system comprises a speech processing part 7 which is applied remote from second part from the hearing prosthesis system which is, at least partly, inserted into a cochlea 1 in the head 9. The second part comprises an in the human cochlea insertable hearing prosthesis 2. The hearing prosthesis 2 comprises light sources 3, a central microelectronic control device 4 and measuring electrodes 5. The central microelectronic control device 4 is connected via electrical wires to the light sources 3 and the measuring electrodes 5. The central microelectronic control device 4 is in wireless communication with the remote speech processing part 7 through a communication channel 11. The communication channel 11 allows for bi-directional communication between the speech processing part 7 and the hearing prosthesis 2. The communication channel 11 can be e.g. a radio transmission channel.

The remote speech processing part 7 can be applied inside or outside of the head 9. At least a microphone device of the remote speech processing part 7 shall be applied outside the head 9 in order to receive ambient acoustic signals. For example, the microphone can be attached to an ear 10 of the human.

The hearing prosthesis 2 is at least partly inserted into the cochlea. For this purpose, the light sources 3 are constructed as a chain of illumination segments 6 which are flexibly connected with each other. As an example, FIG. 1 shows three illumination segments 6. The number of three illumination segments 6 is used only for explanatory purposes. In practice it is suggested to implement a number of 20 to 100 illumination segments, in order to provide enough flexibility and adaptability to the form of the cochlea. The illumination segments 6 may comprise local, distributed sections of microelectronic control equipment for local control of light source bodies. Depending on the size of the central microelectronic control device 4, this device may be also inserted into the cochlea 1 or be placed outside the cochlea.

The illumination segments 6 of the light sources 3 are placed in proximity to hearing neurons 8 of the cochlea 1. This effects in a direct emission of light on the neurons. Further, the measuring electrodes 5 are implanted in the cochlea at the hearing neurons, in order to capture any stimulation of them. Through the measuring electrodes 5 the local total compound action potential (CAP) of the spiral ganglion neurons is detected. This delivers precise information about the physiological reactions to a stimulation of the hearing neuron by the light emission means back to the central microelectronic control device 4.

FIG. 2 shows the remote speech processing part 7 in more detail. The speech processing part 7 comprises a microphone device 20, a central speech processor 21 and a transmission device 22, 23. The transmission device comprises a transmitter 22 and an antenna 23. The microphone device 20 is arranged for receiving ambient acoustic signals 24 and for transforming them into electrical signals. The electrical signals are provided to the central speech processor 21. The central speech processor 21 converts the received audio signals into hearing incitation signals. For this purpose, the central speech processor may transform the audio signals from the time domain into the frequency domain. The central speech processor 21 sends the hearing incitation signals to the transmission device 22, 23, whereby the hearing incitation signals are transferred through the communication channel 11. Further, the electrical energy required for operation of the hearing prosthesis 2 is wirelessly transmitted by the transmitter 22.

FIG. 3 shows the hearing prosthesis 2 in more detail. The light sources 3 and a block 38 of measuring electrodes 5 are located within the cochlea 1. The central microelectronic control device 4 is located outside of the cochlea 1.

The central microelectronic control device 4 comprises a central microcontroller 30 which is connected to receiving means 31, 32. Via the receiving means 31, 32 data are exchanged via the communication channel 11 with the remote speech processing unit 7. The receiving means 31, 32 comprise an antenna 32 and a receiver 31. Hearing incitation signals received from the communication channel 11 are fed by the receiving means 31, 32 into the microcontroller 30. Further, the receiver 31 converts the received radio signals into electrical energy for the supply of the electrical components of the hearing prosthesis 2. The microcontroller 30 is connected with the illumination segments 6 of the light sources 3 via a communication bus 34.

Each illumination segment 6 is equipped with a local microelectronic control device 35 which controls a number of independently controllable light source bodies 36 in dependence from the signals received from the microcontroller 30 via the communication bus 34.

The microcontroller 30 is further connected to an input amplification and multiplexing unit 37. The input amplification and multiplexing unit 37 outputs an analog signal which can be provided to an analog/digital-converter of the microcontroller 30. The input amplification and multiplexing unit 37 is connected to a number of measuring electrodes 5. The electrical signals provided from the measuring electrodes 5 are amplified through discrete amplifiers in the unit 37. The output signals of the amplifiers are selected by a multiplexer with the unit input amplification and multiplexing unit 37 and fed to the output of the input amplification and multiplexing unit 37. Further, the input amplification and multiplexing unit 37 may comprise a signal filter.

With the feed back input from the measuring electrodes 5 and the hearing incitation signals received from the communication channel 11 as master control data, a closed loop control of the stimulation of the hearing neurons 8 by the light emitted from the light sources 3 can be implemented in the microcontroller 30. The output signal of the microcontroller 30 sent to the local microelectronic control devices 35 comprises the stimulation pattern for the light source bodies 36. In the output signal the converted audio information from the microphone device 20, which is divided into the several frequency ranges, as well as the feed back signals from the measuring electrodes 5 is contained.

In a further advantageous embodiment the local microelectronic control devices 35 can be field-programmable gate arrays (FPGA) with logic blocks.

FIG. 4 shows a chain of illumination segments 6 which are connected through flexible bond wires 40 with each other. The bond wires 40 may have a size of 10 μm. Through these bond wires 40 the energy supply for the light emission 36 of the illumination segments 6 and the addressing of the light source bodies 36 is made. For example, with eight wires 256 light source bodies can be independently addressed. Further, the necessary control electronic is implemented on the illumination segments 6 through the microelectronic control device 35 which is integrated on a CMOS substrate through flip-chip bonding.

A further advantageous embodiment of the chain of illumination segments is shown in FIGS. 5 and 6. As already shown in FIG. 4, the single chain of illumination segments causes gaps between the light source bodies 36. In order to bridge these gaps, a double chain of illumination segments 6, 50 is proposed in FIGS. 5 and 6. The double chain can be implemented by placing segments 50 with displacement to segments 6 and with an overlap between the segments 6 and 50. Further, the necessary microelectronic control device 35 can be implemented on a separate connection element 51, which can be implemented as a dice. FIG. 5 shows only a section of the double chain. FIG. 6 shows a double chain inserted in the cochlea 1.

For example, each illumination segment 6, 50 can comprise 100 to 200 light source bodies 36 in the form of micro-LEDs.

FIG. 7 shows a light emission body implemented as a gallium nitride micro-LED. The micro-LED can be manufactured with lateral extensions from 10 to 40 μm.

A gallium nitride-LED comprises a gallium nitride (GaN) layer 81 on a substrate layer 82. The substrate layer 82 is required for compensation of lattice mismatching between the substrate layer 82 and the consecutive layer. The substrate may be manufactured from sapphire, silicon (Si) or silicon carbide (SiC).

On the gallium nitride layer 81 a n-doped gallium nitride layer 80 is provided. An n-contact 75 of the LED is connected to the n-doped gallium nitride layer 80.

Further, the n-doped gallium nitride layer 80 is provided with an aluminium gallium nitride (AlGaN) layer 78. On the aluminium gallium nitride layer 78 a p-doped gallium nitride layer 77 is provided. The p-doped gallium nitride layer 77 is connected to a p-contact of the LED.

When electrical energy is provided to the contacts 75, 76, a recombination of charge carriers occurs in a layer 79 between the n-doped gallium nitride layer 80 and the aluminium gallium nitride layer 78.

The n-contact 75 is connected via a flip-chip bond contact 73 to a connection pad 71 on a CMOS (complementary metal oxide semiconductor) submount 70. Further, the p-contact 76 is connected via a flip-chip bond contact 74 to a contact pad 72 of the CMOS submount 70.

FIG. 8 shows an embodiment for a control of a LED 36. With the control arrangement of FIG. 8 a duration of light emission control can be performed. The LED 36 can be switched via a transistor 83. The transistor 83 is controlled by a one bit register which can be implemented in the form of a flip-flop. The flip-flop has a clock input which is connected through an addressing gate 85 to address lines 86. Through addressing the flip-flop 84 the duration of the on- and off-state of the LED can be digitally controlled.

FIG. 9 shows an embodiment for LED control which allows for control of the intensity of light emission of the LED 36. The LED 36 is connected to a current source circuit 90, 91. The current source circuit 90, 91 comprises a operational amplifier 91 and a resistor 90. The LED 36 is connected between the output of the operational amplifier 91 and its negative input. The positive input of the operational amplifier 91 is connected as a control input to a digital/analog-converter 92. Through the digital/analog-converter 92 the light intensity of the LED 36 can be set to a desired value. Through the current source 90, 91 any nonlinear current/voltage characteristic of the LED 36 can be compensated.

1. In the human cochlea (1) insertable hearing prosthesis (2) with signal emitting means for the stimulation of hearing neurons (8), characterized by the following features:
a) the signal emitting means comprise light sources (3),
b) the hearing prosthesis (2) comprises a microelectronic control device (4, 35), which is arranged for the control of at least one of the intensity, the duration and the repetition frequency of light emission of the light sources (3).

2. Hearing prosthesis according to claim 1, characterized in that through the light sources (3) light is directly emitted on the spiral ganglion neurons in a human cochlea (1).

3. Hearing prosthesis according to claim 1, characterized in that at least one of the intensity, the duration and the repetition frequency of light emission of the light sources (3) is controlled according to audio information to be reproduced.

4. Hearing prosthesis according to claim 1, characterized in that at least a part of the light sources (3) is arranged for the emission of light in the visible wave length range, in particular in the wave length range from 380 to 770 nm.

5. Hearing prosthesis according to claim 1, characterized in that the hearing prosthesis (2) comprises measuring electrodes (5, 38) which are arranged for capturing stimulation signals of the spiral ganglion neurons, whereas the microelectronic control device (4, 35) is connected with the measuring electrodes (5, 38) and the microelectronic control device (4, 35) is arranged for control of at least one of the intensity, the duration and the repetition frequency of light emission of the light sources (3) in dependence of the signals received from the measuring electrodes (5, 38).

6. Hearing prosthesis according to claim 1, characterized in that the light sources (3) are formed as a chain of a plurality of illumination segments (6, 50) which are flexibly connected with each other.

7. Hearing prosthesis according to claim 6, characterized in that the illumination segments (6, 50) are arranged with displacement between neighbouring segments, whereas neighbouring segments are overlapping.

8. Hearing prosthesis according to claim 1, characterized in that the light sources (3) comprise a plurality of light source bodies (36) which are independently from each other controllable by the microelectronic control device (4, 35).

9. Hearing prosthesis according to claim 8, characterized in that one, a plurality or all light source bodies (36) are each equipped with own light focusing means (89).

10. Hearing prosthesis according to claim 8, characterized in that one, a plurality or all light source bodies (36) are each controllable through a controllable current source.

11. Hearing prosthesis according to claim 1, characterized in that the light sources (3) comprise micro-LEDs (88).

12. Hearing prosthesis according to claim 1, characterized in that the light sources (3) are arranged for light emission with different wave lengths.

13. Hearing prosthesis according to claim 12, characterized in that the light sources (3) are arranged for the emission of blue, yellow and/or green light.

14. Hearing prosthesis according to claim 13, characterized in that the microelectronic control device (4, 35) is arranged for controlling the light sources (3) to emit blue light for the activation of a hearing neuron (8), to emit yellow light for the inhibitation of a hearing neuron (8) and to emit green light for the deactivation of a hearing neuron (8).

15. Hearing prosthesis according to claim 1, characterized in that the light sources (3) comprise gallium nitride LEDs (88).

16. Hearing prosthesis according to claim 1, characterized in that the light sources (3) and the microelectronic control device (4, 35) are produced through CMOS gallium nitride flip-chip bonding.

17. Hearing prosthesis according to claim 1, characterized in that the light sources (3) comprise line- and/or matrix-shaped arrangements of a plurality of light source bodies (36).

18. Hearing prosthesis according to claim 1, characterized in that the maximum length of the light sources (3) is less than 30 mm.

19. Hearing prosthesis system comprising:
a) an in the human cochlea (1) insertable hearing prosthesis (2) according to one of the preceding claims, whereas the hearing prosthesis (2) further comprises receiving means (31, 32) for receiving hearing excitation signals,
b) a microphone (20) device for receiving ambient acoustic signals,
c) a transformation unit (21) connected to the microphone device (20), whereas the transformation unit (21) is arranged for transforming the signals received from the microphone device (20) into hearing excitation signals,
d) a transmission device (22, 23) connected to the transformation unit (21), whereas the transformation unit (21) is arranged for transmitting the hearing excitation signals through the transmission device (22, 23) to the receiving device (31, 32).

20. Hearing prosthesis system according claim 19, characterized in that the transformation unit (21) is arranged for the supply of electrical energy the human cochlea (1) insertable hearing prosthesis (2).

21. Method for operating an in the human cochlea (1) insertable hearing prosthesis (2) according to claim 1, characterized in that at least one of the intensity, the duration and the repetition frequency of light emission of the light sources (3) is controlled according to audio information to be reproduced.

22. Method according to claim 21, characterized in that hearing excitation signals are received from a transmission device (22, 23) and at least one of the intensity, the duration and the repetition frequency of light emission of the light sources (3) is controlled according to the received hearing excitation signals.

23. Method according to claim 21, characterized in that for the activation of a hearing neuron (8) blue light is emitted, for the inhibition of a hearing neuron (8) yellow light is emitted and for the deactivation of a hearing neuron (8) green light is emitted.

24. Method according to claim 21, characterized in that independent light source bodies (36) of the light sources (3) are individually controlled in dependence from the frequency range of the audio information to be reproduced.

25. Method according to claim 21, characterized in that stimulation signals of the spiral ganglion neurons are received by measuring electrodes (5, 38) and fed to the hearing prosthesis (2) comprises which are arranged for capturing, whereas the hearing prosthesis (2) controls at least one of the intensity, the duration and the repetition frequency of light emission of the light sources (3) in dependence of the signals received from the measuring electrodes (5, 38) in a closed loop control process.
